(19) United States
(12) Patent Application Publication
(10) Pub. No.: US 2012/0184860 A1
(43) Pub. Date: Jul. 19, 2012

(54) Pulse Diagnosis Instrument

(76) Inventors: Yue Pan, Shanghai (CN); Huilin Zhou, Shanghai (CN); Zhe Pan, Shanghai (CN); Linyan Wang, Shanghai (CN); Weichang Tang, Shanghai (CN)

(21) Appl. No.: 13/143,915

(22) PCT Filed: May 10, 2010
(86) PCT No.: PCT/CN2010/000660

§ 371 Date: Aug. 23, 2011

(30) Foreign Application Priority Data
Sep. 21, 2009 (CN) 200910057921.0

(51) Int. Cl. A61B 5/024

(52) U.S. Cl. 600/500

(57) ABSTRACT

The invention provides a device for sphygmus diagnosis, including a positioning component independently fixed around one's wrist, said positioning component has a visible positioning window. Said sphygmus diagnosis device also has a sensor component which is installed in the positioning component. After assembly the sensor probe of said sensor component is located inside said positioning window. The invention has the following advantages: 1. The determinate position of pulse is accurate. 2. Avoiding the measuring position's deviation caused by the moving of the wrist. 3. Ensuring the stability when positioning component fixed around the wrist. 4. The wrist-pillow can rotate freely, which is convenient to achieve the switch between left and right hand for acquisition for pulse.

### FIELD OF THE INVENTION

The present invention relates to a device for collecting sphygmus (pulse) diagnostic information, in particular to a device for sphygmus diagnosis as this related to traditional Chinese medicine (TCM).

### BACKGROUND OF THE INVENTION

The practitioner of traditional by Chinese medicine (TCM) frequently employs diagnostic methods including inspection, auscultation/olfaction, interrogation, feeling the pulse, among which feeling the pulse is one of the important diagnostic methods. Feeling the sphygmus (pulse) is to detect sphygmus information through tactile pressure receptors (pallesthesia) in the practitioner's finger tips under different pressures (that is floating pressure, medium pressure, heavy pressure) on the arterial sphygmus (pulse). The sphygmus information includes the number of heart beats per minute, rhythm, drifting, pulse strength/thickness, the rigidity or flexibility of the blood vessels, fluency and difficulty and so on. Therefore, the TCM sphygmus information should be interpreted as the collection of the pressure of feeling the sphygmus and the pulsation power of sphygmus wave. The terms “sphygmus” and “pulse” are used interchangeably in this application.

The existing sphygmus detecting devices are usually divided into two kinds, the first kind is to fix the detecting device around one's wrist, for example, the description for utility model of China CN2420975Y (announced on Feb. 28, 2001) disclosed a intelligent TCM sphygmus detecting device (Please refer to FIG. 1), and the description for utility model of China CN2255818Y (announced on Feb. 28, 2001) disclosed a wrist-style probe for electrical sphygmus diagnosis instrument (Please refer to FIG. 2); and the description for invention of China CN2255818Y (announced on Oct. 10, 2007) disclosed a kind of sphygmus manifestation detector for three regions and nine divisions. The practitioner typically detects the desired position for the sphygmus detector on the body and then places the sphygmus detector in that location. One shortcoming of the above-mentioned devices is that the sphygmus detector cannot be accurately aligned on the body because the position of the sphygmus detector on the patient is obscured by the structure of the device.

Another kind of detection device is to fix one's hand in the instrument, and adjust the detecting location through a number of knobs, for example, the description for invention of China CN15653878A (announced on Jan. 19, 2005) disclosed a kind of TCM sphygmus diagnosis instrument. But, the above-mentioned device has a comparatively large size, and wrists and detection device are difficult to maintain in alignment. If the wrist rotates or moves slightly, the best signal cannot be detected.

### SUMMARY OF THE INVENTION

The disclosed pulse diagnosis instrument addresses the shortcoming of the prior art devices to provide a device for sphygmus diagnosis, which aligns the sensor and the sphygmus accurately, and it can maintain a relatively fixed position between the sensor and the wrist, which improves the convenience and accuracy of measurement.

To solve the above technical problems, the present invention provides a device for sphygmus diagnosis, including a positioning component independently fixed around one's wrist, and the said positioning component has a visible positioning window.

Preferably, the said sphygmus diagnosis device also includes a sensor component which is installed in the positioning component. After assembly, the sensor probe of said sensor component is located inside the positioning window.

Further, there are positioning marks around the positioning window for directing the position of sphygmus. Further, said positioning marks are respectively positioned in the middle of each frame of said positioning window. Further, said sensor has an elastic snap-fit, said positioning component has a slot, when assembling, the elastic snap-fit of said sensor component is stuck in the slot of said positioning component. Further, the part of said sensor component fitting people's wrist is a concave arc. Further, on said concave arc there is a medical silica gel membrane layer. Further, in the base of said positioning sensor there are two arc shapes for fixing said positioning component around one's wrist. Further, a nylon-bandage slips over said arc-shaped spring piece. Further, said sphygmus diagnosis device also has a base for propping up one's fore-arm, inside said base there sets a circuit board and said sensor component is connected to said base by data wire. Further, in said base there is a wrist-pillow. The bottom of said wrist-pillow is fixed in said base through a tension spring, and said wrist-pillow can rotate 180 degrees relatively to said base.

In the invention, positioning can be achieved by a visible positioning, and comparatively to the now available sphygmus diagnosis device it has the following advantages:

The positioning process is visible, and the positioning window has tick marks, so the determinate position of sphygmus is accurate.

The positioning component is independently fixed around the wrist, and the position thereof is fixed relatively around the wrist, avoiding the deviation of the measuring position caused by the moving of the wrist relative to the sensor.

The part of said positioning component fitting the wrist is a arc-concave over which slipping a medical silica gel membrane layer, and plus two concave-shaped spring pieces, ensures amenity and stability when positioning component fixed around the wrist.

As the wrist-pillow has a tension spring construction, it cannot be removed from the base during use to avoid losing the pillow, and can rotate freely, which is convenient to achieve the switch between left and right hand for acquisition of sphygmus signal.

### BRIEF DESCRIPTION OF THE DRAWINGS

The disclosed embodiment will now be described in more details as follows combining the drawings with the embodiments.

FIG. 1 is a view of the first kind of a existing sphygmus diagnostic device.

FIG. 2 is a view of the second kind of a existing sphygmus diagnostic device.

FIG. 3 is a view of the third kind of a existing sphygmus diagnostic device.

FIG. 4 is a view of the fourth kind of a existing sphygmus diagnostic device.

FIG. 5 is a schematic structural view of the positioning component.

FIG. 6 is a top view of the positioning component.

FIG. 7 is a front view of the positioning component.

FIG. 8 is a schematic structural view of the sensor component.

FIG. 9 is an embodiment of the device for sphygmus diagnostic.

FIG. 10 is a schematic view of the device for sphygmus diagnostic in use.

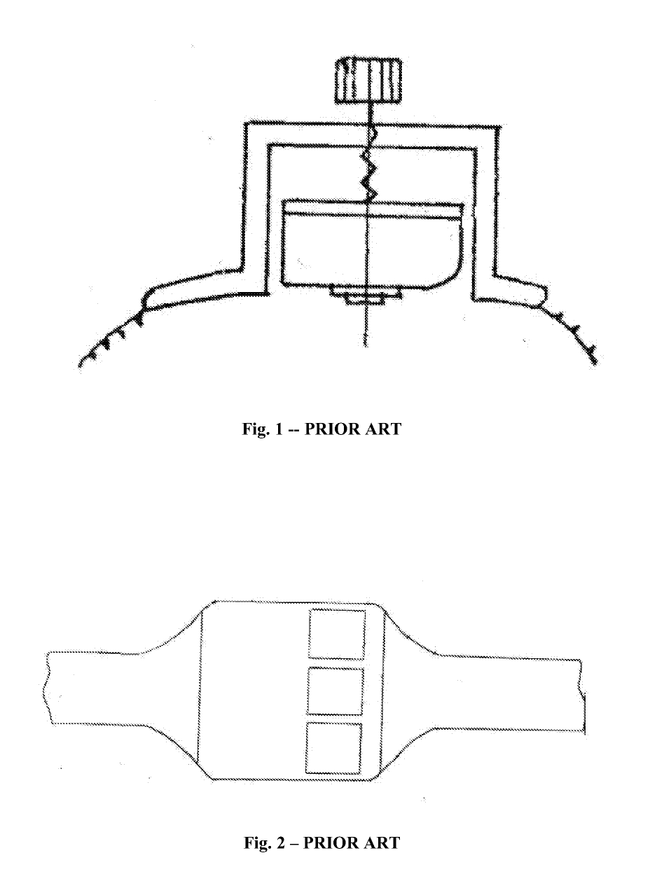

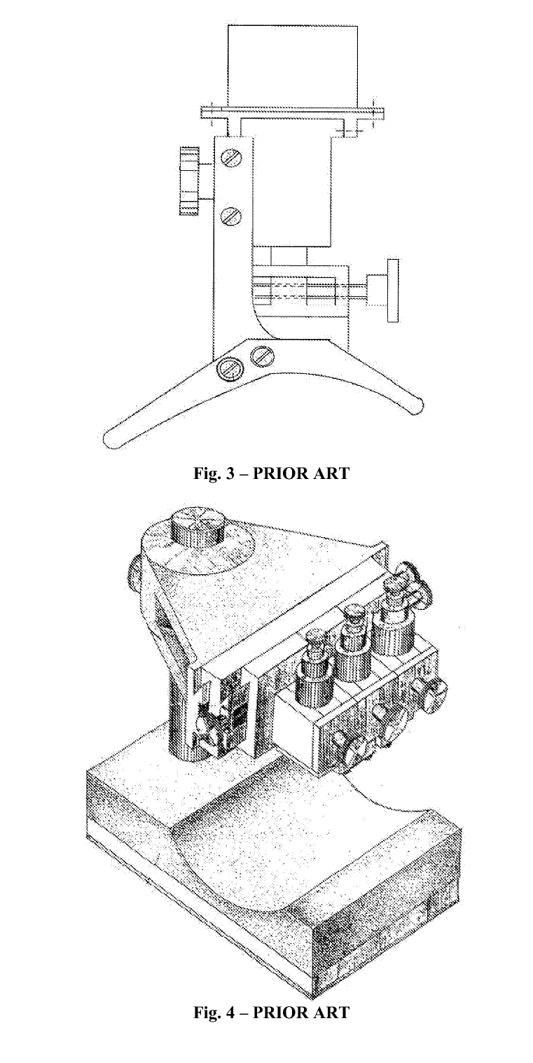

### DETAILED DESCRIPTION OF PREFERRED EMBODIMENTS

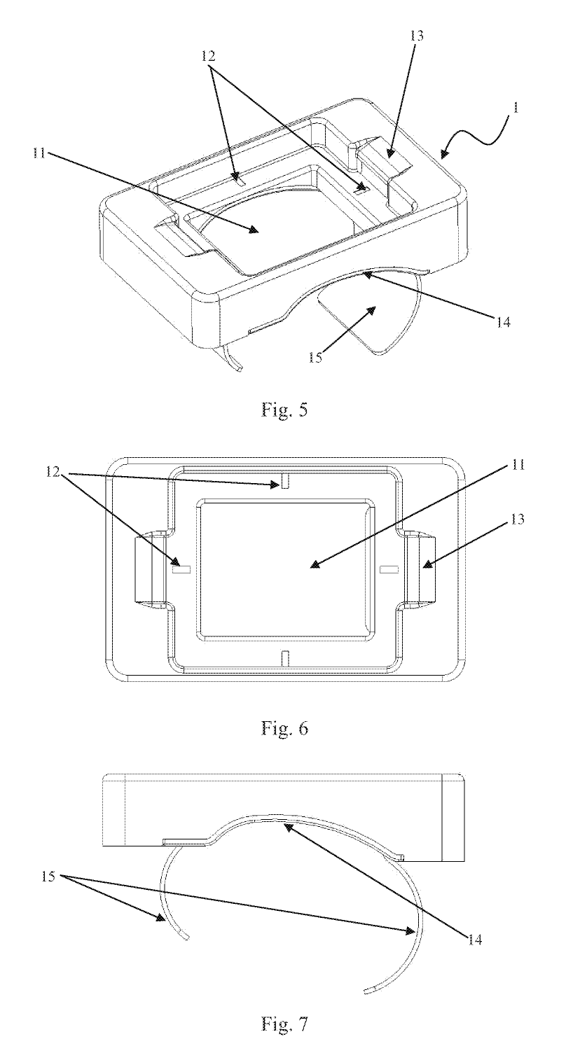

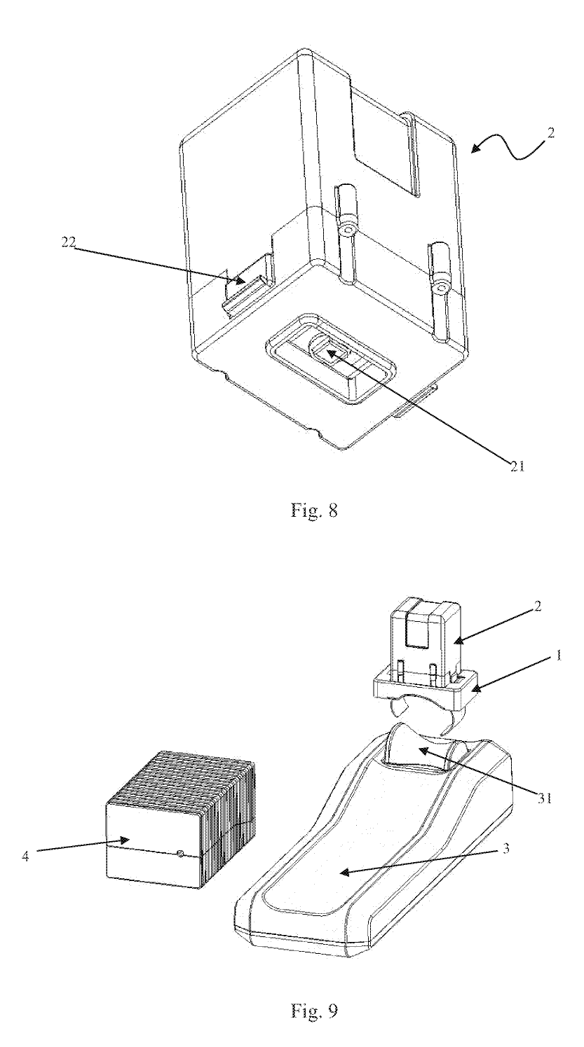

FIG. 5 illustrates one of the embodiments of the sphygmus diagnosis device according to the disclosure, comprising a positioning component 1 fixed independently around the wrist, the positioning component 1 has a visible positioning window 11. The sphygmus diagnosis device also has a sensor component 2 (please refer to FIG. 8). The sensor component 2 is installed in the positioning component 1 as shown in FIG. 9. After assembly, the sensor probe 21 of said sensor component 2 is located inside said positioning window 11.

As shown in FIG. 5 and FIG. 6, around the positioning window 11 there are positioning marks 12 for directing the positioning of the sphygmus sensor. Preferably, positioning marks 12 can be respectively positioned in the middle of each frame of said positioning window 11. Correspondingly, after assembly, the sensor probe 21 is located in the center of the positioning window 11, that is to direct to the position of detected radial artery.

The sensor component 2 has a elastic snap-fit 22, the positioning component 1 has a corresponding cooperative slot 13. When assembling, the elastic snap-fit 22 of the sensor component 2 is stuck inside the slot 13 of the positioning component 1.

The part of positioning component 1 fitting the wrist is a concave arc 14, over the concave arc 14 pastes a medical silica gel membrane layer. In the base of said positioning sensor there are two arc-shaped spring pieces 15 for fixing the positioning component 1 around one's wrist. A nylon-bandage slips over the arc-shaped spring piece 15.

The sphygmus diagnosis instrument also has a base 3 for propping up the patient's fore-arm, according to one of the disclosed embodiments. Inside the base 3 sets a circuit board, the sensor component 2 is connected to the base 3 by data wire. In the base 3 sets a wrist-pillow 31. The wrist-pillow is fixed in the base 3 through a tension spring (not shown), so the wrist-pillow 31 can rotate 180 degrees relatively to the base 3.

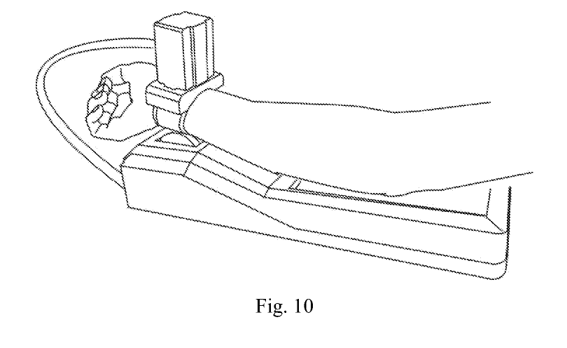

FIG. 10 is an active condition view of the disclosed embodiment, the optimal method for fixing the positioning component 1 is to firstly place the positioning component 1 around the wrist. Then a traditional Chinese doctor places his fingers inside the positioning window to find the accurate position of sphygmus. The doctor then moves the positioning component, so that the positioning marks 12 are centered on the position of sphygmus. The positioning component is then fixed in position through the nylon bandage across the two concave-shaped spring pieces in the base of the positioning component 1.

After fixing the positioning component, the fore-arm is positioned in the base 3 resting on the wrist-pillow 31. The wrist-pillow 31 is designed according to human engineering, when placing one's hand in the wrist-pillow, the tangent plane of the detected radial artery is horizontal, which makes the sensor component vertically installed, and when measuring the right hand and the left hand, the wrist-pillow can rotate 180 degrees for using according the symmetry principle.

1. A device for sphygmus diagnosis, comprising a positioning component independently fixable around the patient's wrist, said positioning component including a visible positioning window.

2. The device as set forth in claim 1, wherein said diagnosis device also has a sensor component which is removably insertable in the positioning component, which after assembling the sensor probe of said sensor component is located inside the positioning window.

3. The device as set forth in claim 2, wherein around said positioning window there are positioning marks for directing to the position of pulse.

4. The device as set forth in claim 3, wherein said positioning marks are respectively positioned in the middle of each side of said positioning window.

5. The device as set forth in claim 2, wherein said sensor has a elastic snap-fit, said positioning component has a cooperating slot, and when assembling, the elastic snap-fit of said sensor component is stuck inside the slot of said positioning component.

6. The device as set forth in claim 2, wherein the part of said sensor component fitting one's wrist is a concave arc.

7. The device as set forth in claim 6, wherein in said arc-concave there pastes a medical silica gel membrane layer.

8. The device as set forth in claim 2, wherein in the base of said positioning sensor there are two arc-shaped spring pieces for fixing said positioning component around the wrist.

9. The device as set forth in claim 8, wherein a nylon-bandage slips over outside said arc-shaped spring piece.

10. The device as set forth in claim 2, wherein said pulse diagnosis device also has a base for propping up the patient's fore-arm, inside said base sets a circuit board, the sensor component is connected to the circuit board by data wire.

11. The device as set forth in claim 10, wherein said base includes a wrist-pillow.

12. The device as set forth in claim 11, wherein the bottom of said wrist-pillow is fixed to said base by a tension spring, and said pulse-pillow can rotate 180 degrees relative to said base.
